(19) United States
(12) Patent Application Publication
(10) Pub. No.: US 2011/0040371 A1
(43) Pub. Date: Feb. 17, 2011

(54) COILED ASSEMBLY FOR SUPPORTING THE WALL OF A LUMEN

(75) Inventors: Johannes Hendrikus Leonardus Hanssen, Erlecom (NL); Petrus Antonius Besselink, Enschede (NL)

Correspondence Address: TRASKBRITT, P.C., P.O. BOX 2550, SALT LAKE CITY, UT, 84110, US

(73) Assignee: HANSSEN INVESTMENT & CONSULTANCY B.V., Erlecom (NL)

(21) Appl. No.: 12/735,877

(22) PCT Filed: Feb. 20, 2009
(86) PCT No.: PCT/EP2009/052041

§ 371 Date: Oct. 20, 2010

Related U.S. Application Data
(60) Provisional application No. 61/030,621, filed on Feb. 22, 2008.

(51) Int. Cl. A61F 2/82

(52) U.S. Cl. 623/1.22

(57) ABSTRACT

The invention is in the field of mechanical devices. It relates to a device and methods for supporting the wall of a lumen. In particular, the invention relates to a device and method for supporting the wall of a human or animal body lumen, the device comprising a coiled body which can be inserted into a lumen and expand therein. The device may advantageously be used for treating arterial and vascular diseases, in particular coronary conditions and cardiovascular diseases. The device may also advantageously be used for the localized delivery of drugs. In particular, the invention provides a device for supporting the wall of a lumen comprising a coiled structure (10) defining an inner core (20) wherein the inner core comprises a support wire (30) comprising a shape memory material, wherein the shape memory material is programmed to make the coiled structure (10) assume a macro-coiled structure (40) upon an appropriate trigger.

### FIELD OF THE INVENTION

The invention is in the field of mechanical devices. It relates to a device and methods for supporting the wall of a lumen. In particular, the invention relates to a device and method for supporting the wall of a human or animal body lumen, the device comprising a coiled body which can be inserted into a lumen and expand therein. The device may advantageously be used for treating arterial and vascular diseases, in particular coronary conditions and cardiovascular diseases. The device may also advantageously be used for the localized delivery of drugs.

### BACKGROUND OF THE INVENTION

Coiled wires, suitable for keeping a body lumen open have been described. U.S. Pat. No. 6,086,547 describes a coiled guide wire for medical use, in particular for diagnostic purposes such as catheterization. During the diagnostic examination the guide wire is introduced into for example the patient's vascular system, and the smooth outer surface of the guide wire ensures that the tissue, in particular the walls of the blood vessels, is not damaged.

WO 9742910 describes a manufacturing processes for an apparatus, including a slotted hypotube, for use as a catheter, a guidewire, a catheter sheath for use with catheter introducers or a drug infusion catheter/guidewire. The manufacturing process includes creating a pattern of slots or apertures in a flexible metallic tubular member, by processes including electrostatic discharge machining (EDM), chemical milling, ablation and laser cutting. These slots or apertures may be cut completely or partially through the wall of the flexible metallic tubular member.

WO97/38730 describes the use of a stent in the form of a spring for the delivery of radioactive material to a precise location in the human body.

WO 98/29148 discloses a multilayer device comprised of at least one layer of material which is capable of absorbing liquid to thereby increase the volume of the layer, i.e., liquid swellable, and when bound to at least one non-absorbing or lesser absorbing layer of material causes deformation of the device upon liquid absorption. It is disclosed that the device may also be formed as a spiral or spring.

WO98/23228 relates to a directional drug delivery stent which includes an elongated or tubular member having a cavity containing a biologically active agent. In one embodiment, the active agent is diffused from the reservoir directly to the walls of a body lumen, such as a blood vessel, through directional delivery openings arranged on an outer surface of the elongated member. Another variation of the stent includes an osmotic engine assembly for controlling the delivery of the active agent from the reservoir. The drugs which may be applied by the directional delivery stent include steroids, anti-inflammatory agents, restenosis preventing drugs, anti-thrombotic drugs, and tissue growth regulating drugs.

In many stents there is also a drug-eluting layer applied to the surface. There is a limitation, however, to the thickness of the active layer and therefore to the possible maximum drug output.

Pijls et al., (Eur. J. Pharm. Biopharm. 59, 283 (2005)) describe a particular drug-eluting device, called the OphthaCoil, which consists of a thin metallic wire, which is coiled and carries a drug-loaded adherent hydrogel coating on its surface. The drug is then released in a more or less controlled fashion to the anterior side of the eye.

The drug loading capacity of the OphthaCoil is very limited however.

Several strategies have been suggested to solve this problem. First, it was suggested to fill the coil with a hydrogel. When polymerizing a hydrogel in the lumen itself, it appeared that the coil lost its flexibility. The loss of flexibility is detrimental for the patient, since the device is no longer tolerated in the eye when rigid.

Secondly, it was attempted to insert a number of straight wires, made of the same material as the wires constituting the coil, into the coil. The straight wires were coated with the same coating as the coil and so increased the drug load of the assembled device. The coils thereby lose some of their flexibility by that process (Pijls et al, supra).

WO2007006427 describes a third improvement to the coiled wire for the controlled release of drugs to the eye. Herein, the drug releasing characteristics of the device were greatly improved by introducing micro-particles such as micro-spheres or microbeads that contain the drug of choice into the lumen of the coil.

Another attempt to increase the drug loading capacity of a coiled stent is described in EP 1117351. Therein a drug delivery device is disclosed that includes a helical body having a plurality of coils and an interstice that occurs between the coils and wherein a therapeutic or diagnostic agent is carried within the interstice to be delivered to a biological tissue including muscle tissue, neoplastic tissue, vascular tissue, or any other type of body tissue. Said helical structure may be manufactured from a biocompatible straight wire. A particularly useful embodiment of this invention however, is found wherein the helical body comprises a spring formed into a plurality of coils to provide said helical body.

Such a device, carrying a double coiled wire, may be made of any flexible material and can even be manufactured from a shape-memory material, in order to have it assume a predetermined shape when placed in the body (the wound state) whereas in the unwound state it may assume a stretched shape in order to facilitate the placement of the device through a stenting procedure, such as for instance using a guidewire.

Particular disadvantage of such devices is that commonly used materials, such as shape-memory materials show symptoms of fatigue, making such devices less suitable for implantation at sites exposed to mechanical stress. When placed in arteries, stents are subject to considerable radial forces, in particular in coronary arteries, whereas when placed in larger vessels for instance in the joints, the stent may be exposed to considerable axial forces.

Such doubly coiled helical devices made of shape memory material would then run a considerable risk of breaking and thereby damage the artery or blood vessel wall.

### SUMMARY OF THE INVENTION

The invention relates to an improved device for supporting the wall of a lumen. A device according to the invention has an improved flexibility, improved fatigue behavior and a good supporting function. The invention accordingly provides a device for supporting the wall of a lumen comprising a coiled structure (10) defining an inner core (20) wherein the inner core comprises a support wire (30) comprising a shape memory material, wherein the shape memory material is programmed to make the coiled structure (10) assume a macro-coiled structure (40).

The invention also provides a method for supporting the wall of a lumen by inserting a device according to the invention into the lumen, inducing the support wire assuming a predetermined shape, thereby expanding the device in the radial and/or axial direction.

### DETAILED DESCRIPTION OF THE INVENTION

The invention relates to an improved device for supporting the wall of a lumen. A device according to the invention has an improved flexibility, improved fatigue behavior and a good supporting function. The invention accordingly provides a device for supporting the wall of a lumen comprising a coiled structure (10) defining an inner core (20) wherein the inner core comprises a support wire (30) comprising a shape memory material, wherein the shape memory material is programmed to make the coiled structure (10) assume a macro-coiled structure (40).

It was surprisingly found that the combination of a flexible coiled structure, supported by the shape memory support wire inside, greatly improved the fatigue behavior of the assembly.

A shape memory material or shape memory alloy (SMA, also known as a “smart metal”, “memory alloy”, or “muscle wire”) is an alloy that “remembers” its geometry or shape. The memory metal may assume its final structure spontaneously in that it returns to a “programmed” shape after being deformed. The shape memory material may also return to its programmed structure by applying an appropriate trigger, usually heat, to the alloy. Shape memory materials are already commonly used in hydraulic, pneumatic, and motor-based systems. Shape memory materials also have numerous applications in the medical and aerospace industries.

Hence the invention also relates to a device as described above wherein the shape memory material is programmed to make the coiled structure (10) assume a macro-coiled structure (40) upon the application of an appropriate trigger. Such a trigger may be an internal trigger such as the patient's own body heat or an external trigger such as an ultrasound trigger applied to the body using an external source.

The three main types of SMA are the copper-zinc-aluminum-nickel, copper-aluminium-nickel, and nickel-titanium (NiTi) alloys. Repeated use of the shape memory effect may lead to a shift of the characteristic transformation temperatures (this effect is known as functional fatigue, as it is closely related with a change of microstructural and functional properties of the material).

Shape memory alloys may have different kinds of shape memory effect. The two most common memory effects are the one-way and two-way shape memory. When a shape memory alloy is in its cold state, the metal can be bent or stretched into a variety of new shapes and will hold that shape until it is heated above the transition temperature. Upon heating, the shape changes back to its original shape, regardless of the shape it was when cold. When the metal cools again it will remain in the hot shape, until deformed again.

With the one-way effect, cooling from high temperatures does not cause a macroscopic shape change. The two-way shape memory effect is the effect that the material remembers two different shapes: one at low temperatures, and one at the high temperature shape. A material that shows a shape memory effect during both heating and cooling is called two-way shape memory. This can also be obtained without the application of an external force (intrinsic two-way effect). The reason the material behaves so differently in these situations lies in training. Training implies that a shape memory can “learn” to behave in a certain way. Under normal circumstances, a shape memory alloy “remembers” its high-temperature shape, but upon heating to recover the high-temperature shape, immediately “forgets” the low-temperature shape. However, it can be “trained” to “remember” to leave some reminders of the deformed low-temperature condition in the high-temperature phases.

This allows the metal to be bent, twisted and pulled, before reforming its shape when released. This means that the material is often considered as being “nearly indestructible” because it appears that no amount of bending will result in permanent plastic deformation. Practically, the strains are limited to 8% before plastic deformation occurs. When a shape memory material is deformed at temperature above the transformation temperature it can be deformed up to 8% and still return elastically to its undeformed state. This behaviour is called pseudo-elasticity or superelasticity and it occurs with a typical non-linear stress-strain characteristic, which shows different loading and unloading plateaus with a hysteresis.

If a superelastic material, for example NiTi, is deformed a typical loading plateau (stress) is in the range of 400-500 MPa. Typical unloading plateau stresses are in the range of 100-250 MPa.

There is another type of SMA called a ferromagnetic shape memory alloy (FSMA), that changes shape under strong magnetic fields. These materials are of particular interest as the magnetic response tends to be faster and more efficient than temperature-induced responses.

Metal alloys are not the only thermally-responsive materials; shape memory polymers have also been developed, and became commercially available in the late 1990s.

Many metals have several different crystal structures at the same composition, but most metals do not show this shape memory effect. The special property that allows shape memory alloys to revert to their original shape after being triggered is that their crystal transformation is fully reversible. In most crystal transformations, the atoms in the structure will travel through the metal by diffusion, changing the composition locally, even though the metal as a whole is made of the same atoms. A reversible transformation does not involve this diffusion of atoms, instead all atoms shift at the same time to form a new structure. At different temperatures, different structures are preferred and when the structure is cooled through the transition temperature, the martensitic structure forms from the austenitic phase.

Shape memory alloys are typically made by casting, using vacuum arc melting or induction melting. These are specialist techniques used to keep impurities in the alloy to a minimum and ensure the metals are well mixed. The ingot is then hot rolled into longer sections and then drawn to turn it into wire.

The way in which the alloys are “trained” or “programmed”depends on the properties wanted. The “training” dictates the shape that the alloy will remember when it is heated. This occurs by heating the alloy so that the dislocations re-order into stable positions, but not so hot that the material re-crystallizes. They are usually heated to between 400° C. and 500° C. for 30 minutes. Typical variables for some alloys are 500° C. and for more than 5 minutes. They are then shaped while hot and are cooled rapidly by quenching in water or by cooling with air.

The copper-based and NiTi (nickel and titanium)-based shape memory alloys are considered to be engineering materials. These compositions can be manufactured to almost any shape and size. The yield strength of shape memory alloys is lower than that of conventional steel, but some compositions have a higher yield strength than plastic or aluminium.

One of the advantages to using shape memory alloys is the high level of recoverable plastic strain that can be induced. The maximum recoverable strain these materials can hold without permanent damage is up to 8% for some alloys. This compares with a maximum strain 0.5% for conventional steels.

The late 1980s saw the commercial introduction of Nitinol as an enabling technology in a number of minimally invasive endovascular medical applications. While more costly than stainless steel, the self expanding properties of Nitinol alloys manufactured to BTR (Body Temperature Response), have provided an attractive alternative to balloon expandable devices. On average, 50% of all peripheral vascular stents currently available on the worldwide market are manufactured with Nitinol. It is known however, that Nitinol stents suffer from fatigue making them unsuitable for applications wherein high stress is applied to the device, either in axial or in radial directions of the stent.

A device according to the invention suffers less from the negative consequence of fatigue as mentioned above since the memory shape material is situated inside the core of the coiled structure, whereas the shape memory material can still induce the device to take a predetermined shape upon application of the appropriate trigger.

The inner core of the coiled structure may also comprise a drug-eluting material in order to further improve the usefulness of the device in treating disease symptoms such as for instance restenosis. Hence, the invention also relates to a device as described above wherein the inner core comprises a drug-containing layer (21).

The coiled structure may preferably be a coiled spring. The pitch of the spring windings may then determine the rate at which the drug elutes. If the coiled structure consists of windings with different interstitial openings, each section of the coiled structure may have different mechanical properties. Moreover, the different pitch between the windings will also influence the speed of the release drugs 22 from core 20. This is depicted in FIG. 5.

The coiled structure may be made of bare metal, but can also be provided with a coating of a different material including polymers, biodegradable material, a metal, a radio-opaque layer or combinations thereof.

For certain application it is advantageous when the outer surface of the device has certain specific properties. For instance, when the device is used as a stent or for delivering drugs to certain sites of the human or animal body, the outer surface may advantageously be hydrophilic and/or slippery. To that effect, the surface of coil 10 may be coated with a polymer that improves its hydrophilicity.

The outer surface of the coiled structure may also be coated with a drug. This may be done by simply applying the drug onto the surface of the device, the drug may also be contained in a coating applied to the surface of the device. Alternatively, the coiled structure may be made of a biodegradable material containing the drug.

In another embodiment of the invention, the coiled structure consists of a tube, perforated with holes or slots by laser cutting, etching, grinding or any other means. Also, both the support wire and the coiled structure may be made from such perforated tubing, or only one of them. The inner lumen of the perforated tubing may be filled with drugs, which will elute out gradually. It is clear that such tubing has an increased capacity to contain drugs as compared with solid wire or ribbon.

It is a further object of the invention that by variation of the length of the device the gap between the coil windings (pitch) can be adjusted in order to modify the drugs release profile. Such a length change can for example be achieved by wrapping the coiled structure around a balloon catheter, then bring it into place and inflate the balloon to place the device, while the expansion of the balloon causes the pitch of the coils to increase.

The coiled structure may also comprise additional wires in its inner core. Such wires may then provide other characteristics to the device, either in form or function. For instance, the inner core may also contain an additional wire comprising a biodegradable material in order to provide mechanical or functional properties that change over time. The additional wire may also contain a drug that releases into the body, during or after the device is placed.

Accordingly, the invention provides a device as described above comprising an additional wire in the inner core of the coiled structure wherein the additional wire has a property selected from a group consisting of superelasticity, fatigue characteristics, shape memory behaviour, improved radio-opacity, improved stiffness, easy plastic deformation, MRI compatibility and combinations thereof. Such wires can be made of metals, metal alloys like stainless steel, nitinol, cobalt chromium, MP 35N, polymers, drug-eluting polymers and combinations thereof.

Also, an additional wire may be biodegradable, while a second (or third) additional wire may have a permanent life. The biodegradable wire can play a role in the controlled drugs release, if it has been made from a material (e.g. a polymer or a magnesium alloy) that contains drugs on its surface or inside it.

In another embodiment of the invention the additional wire may even contain elements capable of corresponding with a device outside the patient, like an electrical lead in connection with the outside world, which enables the operator to send a signal to the device, such as an electrical or temperature trigger to a shape memory material, or trigger the start of drug release by electrically influencing the behaviour of the drug containing substance 21

In some existing devices there are problems if a biodegradable part is dissolving, because it may break into pieces. This is a typical risk for biodegradable devices that do not have a stable outside structure. An advantage of a device according to the invention is that if a biodegradable core wire is surrounded with a non-biodegradable coil spring that holds everything inside, such pieces could not do harm to the patient. Therefore the coil spring acts as a safe housing for the bio-active components which are placed inside it.

The function of the coiled structure 10 in that case is then to keep the dissolving additional wire inside the inner core 20 for safety reasons. This prevents that fragments of the dissolving centre wire can translocate from the desired location by the blood stream or by any other reason.

The inner core may also comprise a drug-eluting material (21) next to the support wire (30) and optionally the additional wires. The drug-eluting material may partly or entirely fill up the residual space (20) of the inner core.

The device may contain any suitable type of drug. If used for stenting, the device may advantageously contain anti-proliferative drugs such as mTOR blockers, such as sirolimus, taxol, zotarolimus, paclitaxel, everolimus tranilast or analogues thereof.

The influence of the pitch, i.e. the gap between adjacent windings of the coiled structure and therefore the speed of drugs release can also be controlled by the choice of the geometry of the coiled structure. If this structure is made of a wire with a circular cross section, there will be much more interstitial space available than when the structure is made of a flat rectangular ribbon that has its largest length parallel to the length axis of the coiled structure. Of course this also has influence on the mechanical behaviour of the coiled structure. In axial direction it will become more rigid, while the inner space in such a coil spring is larger than the inner space of a coil spring of the same outer diameter, but made from a round wire with the same cross section area as the rectangular ribbon.

Also, the coiled structure can be of different geometries. It can for example also be made of a multiple coil spring made of two, three or more wires that are coiled together, or it can be made of a braided or knitted structure.

It is an object of the invention that by a proper dimensioning of the supporting wire, this wire acts as a framework that holds the assembly in place in the lumen of the patient's body that has to be treated.

A particularly advantageous embodiment of a device according to the invention would consist of a coiled structure in the form of a spring with a first support wire comprising a shape memory material in its inner core with super-elastic and/or shape memory characteristics, which has been programmed to take a predetermined expanded shape after the release of the device in the body lumen in order to provide the device with a perfect fit to the wall of the lumen.

The device may assume all kinds of structures programmed by the wire comprising the shape memory material. This is indicated herein as macro-coiled structure. In the accompanying drawings this is illustrated and exemplified, wherein the macro-coiled structure assumes the shape of a spring. It will be clear for the skilled person that the macro-coiled structure can have different geometries as well. For example, it may be straight, zigzag or have any other geometry.

A number of exemplary structures for the macro-coiled structure is provided in FIG. 5. The superimposed zigzag pattern of FIG. 5A makes the helix less rigid than the one in FIG. 2 and it also increases the effective surface that comes in contact with the surrounding tissue. As can be seen, the wavy support wire has different amplitudes in its zigzag-pattern and it also has different diameters. Together with another parameter, the pitch, many mechanical characteristics can be influenced and thus optimized. It will be clear for the skilled person that for example the radial stiffness, but also the axial flexibility, expansion ratio and fatigue behavior, amongst others, may be controlled and optimized by choosing the right parameters per application. The wavy helix of FIG. 3A is relatively rigid, as the amplitude of the zigzag-pattern is small, but it is more flexible than the helix of FIG. 1B.

By raising the amplitude, a more flexible helix can be made, like the one of FIG. 3B. The most flexible helix is given in FIG. 3C, where a large amplitude is used in combination with a thinner support wire. In the helix of FIG. 3C the pitch is also made smaller than in FIGS. 3A and 3B in order to create more contact surface with the surrounding tissue. Varying these parameters provide the skilled person with a multitude of design opportunities.

In a particularly advantageous embodiment, the support wire may be made of a material, for example nitinol, which has the tendency to take the helical shape as shown, even if it is delivered through a catheter in a straight state.

This means that such a helix regains its shape as soon as it leaves the restraining catheter. This minimizes the delivery profile of such a device. The coiled structure can also be made of a shape memory polymer or alloy with superelastic or shape memory characteristics, a metal alloy like stainless steel, MP 35N, cobalt chromium, polymer or any other material.

If the support wire is made of a material or shape that allows the support wire to vary in length, the overall length of the device can be changed as well. This means that also the coiled structure will change length and the pitch of the adjacent coil windings will thus increase. This makes the openings between those windings larger and the amount of drugs that can pass through these openings can be controlled in such a way. Dependent on the need for a specific patient, the physician can choose for a customized drug-elution profile by increasing or decreasing the length of the coiled structure.

An example of a support wire that can change its length is for example a wire that is constituted, at least partially, of a spring, such as a coiled spring FIG. 4c). The support wire may also comprise an elastic material. A support wire that can increase or decrease its length is herein further referred to as an expandable support wire.

An expandable support wire can for instance comprise stainless steel or cobalt chromium, which can easily be plastically deformed. Inside the expandable support wire there is another space available for placing drugs, which will be released when the device is placed into the patient and when the expandable support wire is stretched. Such may for instance be accomplished by applying balloon pressure.

In a preferred embodiment, a device according to the invention comprising an expandable support wire is wrapped around a deflated balloon, brought into place on a catheter which holds the balloon and then delivered by inflating the balloon. If desirable, the balloon may be deflated and removed, but in other cases it may stay in place for as long as the treatment has to last. Removal of balloon and drug-eluting device together is easy if the coiled structure exhibits elastic deformation. In that case the springs will revert to the shorter length after deflation of the balloon and can be easily removed together with the balloon catheter as soon as the treatment is completed.

In case the balloon has to stay in place for a relatively long period, a balloon with a perfusion channel may advantageously be employed in order to ensure sufficient blood flow during the entire procedure.

In addition to typical stenting applications, where the radial force of the device is used for supporting the surrounding tissue, devices according to this invention may also exclusively be used for drugs release, where radial forces to the tissue are kept as low as possible. Of course some radial force is always needed to keep the device in place. However, devices according to the invention may also be kept in place by separate anchoring devices which are attached to the coiled structure 10 or the macro-coiled structure 40. Typically, such anchoring sections may be an integral part of the device, where locally a higher radial force is active than in the remainder of the device. Such anchoring sections can have a locally thicker support device, a different pitch, a different material, a different heat treatment, anchoring hooks or any other adaptation to provide the required radial force to keep the device in place.

Optionally, such a device may be provided with hooks or brush hairs on the outer surface to improve the grip and prevent migration during use of the device. A device according to the invention may also be used as a so-called AAA stent, this is a bifurcated stent used for the treatment of aortic or abdominal aneurisms.

For specific uses, such as for urinary tract stents, it may be desirable to remove the stent after some prolonged placement. In that case it is desirable to prevent in-growth of cells into the interstitial spaces of the outer coil. For that purpose, a cover or a coating may be applied to the outer coil to prevent such in-growth. The cover and/or coating may comprise polytetrafluorethene (PTFE).

Since the device provides several compartments to store drug containing material, there may be different types of drug containing material stored in the device. This is exemplified in FIG. 4A where reference signs 21 and 22 refer to two different types of drug containing matter contained in different compartments of the device.

The device may contain a drug-containing material both in the inner core 20 of the coiled structure 10 as well as in the core 39 of the windings of an expandable support wire 30 (FIG. 4C). Elution of the drug may now be controlled on multiple levels. Drug containing material in core 39 may contain a different drug as the material contained in core 20 or it may contain the same drug, optionally in different concentrations.

If there is a need to release more drugs for a specific patient, compartment 39 could be filled with a material containing a high concentration of drugs. By stretching the expandable support wire 30 into a deformed state, the length of the device increases and the distance between adjacent windings of the spring comprised in the expandable support wire causes that additional drug will be released from the core of the expandable support wire. This stretching may be caused by remote triggering of a shape memory effect, as described above. Of course the stretching can also be caused by placing the device on a balloon and inflating the balloon while it is in the patient's body.

The device may be used in any lumen of cavity, such as a body lumen or cavity. This may include the vascular system, such as for instance blood vessels in or near the heart, brain and its periphery; the lymph system, the tracheal system; the intestinal tract; the urinary system; the gastroenterological system and the esophagus.

A particularly advantageous use of a device according to the invention is the treatment of an aneurism, in particular a neurological aneurism. A support wire 10 may be programmed to make the coiled structure 20 assume a macro-coiled structure that precisely fits the lumen of the aneurism.

For specific applications the drugs may not be needed and the device is only used for supporting purposes, either permanent or temporary.

Fields of use of devices according to the invention can be in the combined stenting and/or drug delivery in for example gastroenterology, such as for the temporary delivery of drugs in the intestines of patients with Crohn's disease. It may also be used for peripheral or esophageal stenting and even for controlled anticonception by implanting a device, for instance in the uterus. It may also be used in procedures for the delivery of local anesthetics.

A typical example would be a coiled stent that is placed into the intestinal tract on or in a catheter, while it is in a state with reduced diameter. This can be achieved in several ways. The entire device can be collapsed and brought into such a delivery catheter, or it can be mounted on the surface of the delivery catheter while its ends are held tight by some release mechanism.

In both cases the device is brought into the correct position and then released to change shape and fit to the wall of the intestinal tract. In the first case this shape change can take place as soon as the constraining force of the surrounding delivery catheter disappears when the device is pushed out of this catheter. In the second case the ends of the device are released by causing the release mechanisms to let go the ends of the device.

### BRIEF DESCRIPTION OF THE FIGURES

FIG. 1a shows a longitudinal section of a coiled structure 10, with windings 11, which defines an inner core 20 comprising a support wire 30. The support wire comprises a shape memory material programmed to make the coiled structure (10) assume a macro-coiled structure (40) along a longitudinal axis (50) upon an appropriate trigger. This is depicted in a side view in FIG. 1B.

FIG. 2 shows a longitudinal section of a preferred embodiment of the invention wherein the inner core 20 contains a layer of drug-containing material 21. When the device is placed in the patient, the drug-containing material 21 will gradually release the drug, which will then move outward through the gap between the coil windings 11 of coiled structure 10.

FIGS. 3A-C show three different embodiments of a macro-coiled structure. Apart from the helical structure shown in FIG. 1b, a zigzag-pattern may be superimposed on the coiled structure 10 around the support wire 30. Also, a support wire programmed to assume a wavy structure may advantageously be employed to make the macro-coiled structure 40 assume the structures exemplified in FIG. 3B or 3C. The outer diameter of the three examples of FIGS. 3A-C is identical, but their mechanical behavior is very different.

FIG. 4A shows a cross section of a coiled structure 10 in a drug-eluting device. The outer layer is constituted by coiled structure 10, surrounding a drug-containing layer 21 in its core. Layer 21 may elute the drugs itself or optionally be of a porous material which contains other elements inside. In this example a support wire 30 comprising a shape memory material is not the only additional material in the core of the coiled structure. Besides 30, there is also an additional wire 31, which may be for example a radio-opaque material or another shape memory alloy. Another wire 32 is shown, which may consist of a biodegradable material that contains an active component 22, which is embedded in 32 and which may elute while in the patient's body.

FIG. 4B shows several alternative embodiments for support wire 30. Support wire 30 may have a triangular cross-section or a square or rectangular cross-section. These embodiments are particularly advantageous when the support wire 30 comprises a coating on its surface, such as a drug-eluting coating. In that way the coating will better adhere to the surface of support wire 30 and not scrape off when support wire 30 moves in the axial direction through the windings 11 of the coiled structure 10.

FIG. 4C shows a device with a coiled structure 10, having in its core 20 an expandable support wire 30 in the form of a spring defining an inner core 39. The device may contain a drug-containing material both in the inner core 20 of the coiled structure 10 as well as in the core 39 of the windings of an expandable support wire 30.

FIG. 5 shows a longitudinal section of another type of device according to the invention, wherein coiled structure 10 may comprise different types of wire. Wire 12 has a rectangular cross section with a large length-width ratio, whereas wire section 13 has a smaller ratio and section 14 is a square wire.

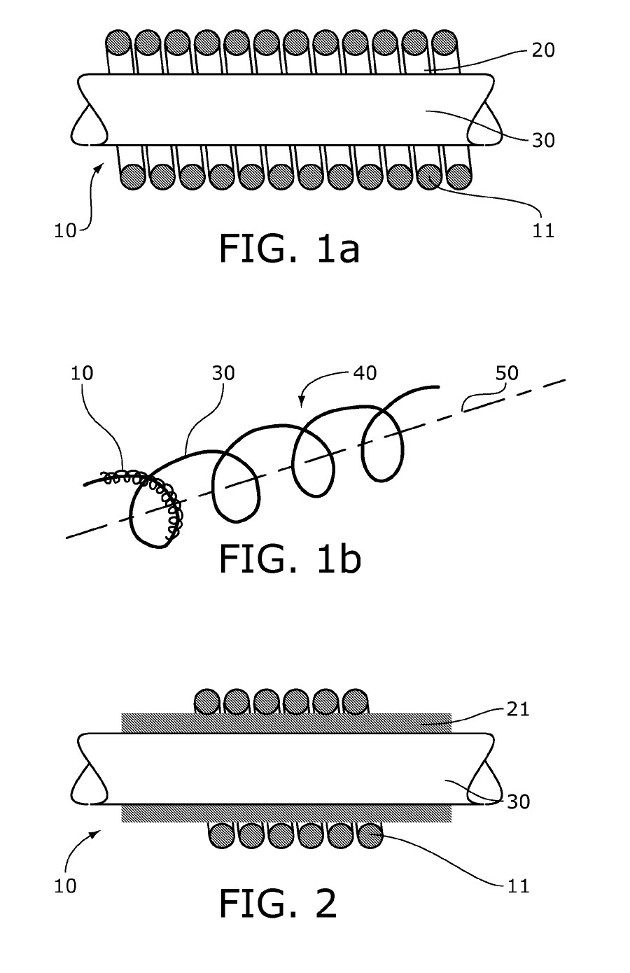

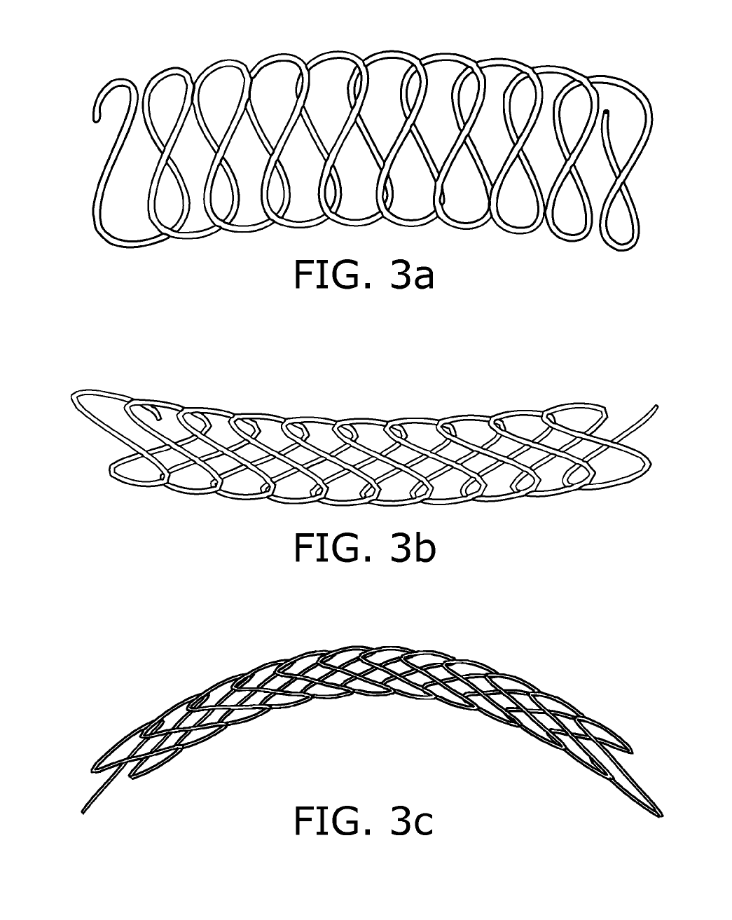

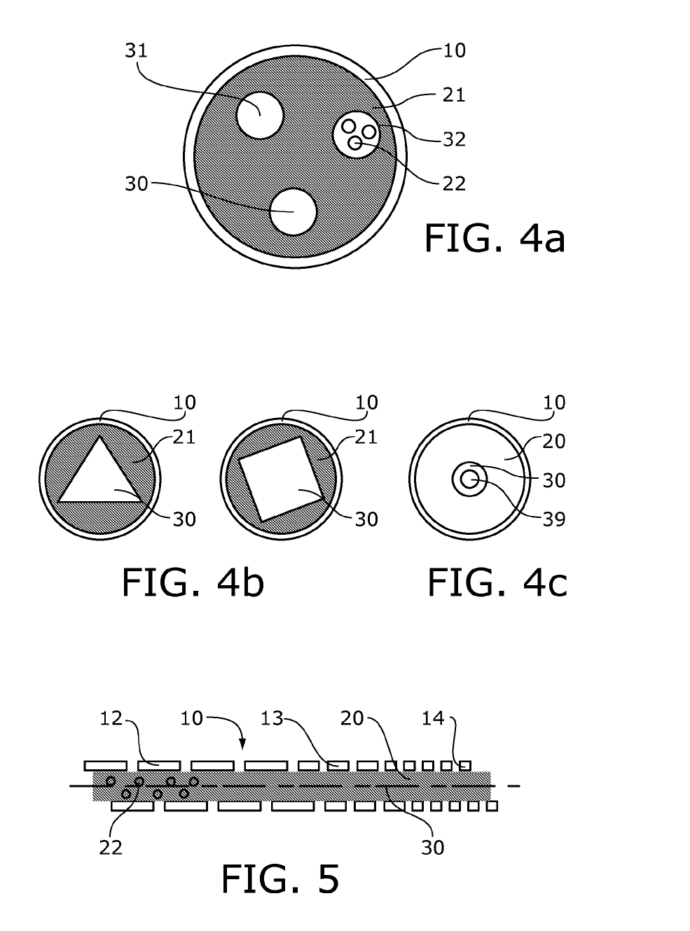

### EXAMPLE

A straight superelastic Nitinol wire with a diameter of 0.2 millimeter and a length of 2.5 meter (Fort Wayne Metals) was coiled into the desired shape around a mandrel, heated to 480 degrees Celsius for 10 minutes and left to cool. This programs the wire to assume the structure defined by the mandrel.

A straight stainless steel 304V wire of 50 micrometer diameter (Fort Wayne Metals) was coiled on a mandrel to obtain a spring with an internal diameter of 0.3 millimeter and a length of 40 centimeters.

If desired, the Nitinol wire was coated with a drug according to standard techniques, basically as described in Hanssen et al., J. Biomed. Mater. Res. 48: 820-828 (1999) which is hereby incorporated by reference herein.

The Nitinol wire was hung vertically and straightened by weight at its bottom end, and the stainless steel spring was slid over the Nitinol wire. The device was cut into the appropriate length and the Nitinol wire was linked to the coiled structure using a UV curable glue. When the weight was released from the Nitinol wire, the device assumed a spiral structure defined by the mandrel of the Nitinol wire.

1. A device for supporting the wall of a lumen, the device comprising; a coiled structure (10) defining an inner core (20) wherein the inner core comprises a support wire (30) comprising a shape memory material, wherein the shape memory material is programmed to make the coiled structure (10) assume a macro-coiled structure (40).

2. The device of claim 1, wherein the coiled structure assumes a macro-coiled structure upon an appropriate trigger.

3. The device of claim 1 or 2, wherein the inner core comprises a drug-containing layer (21).

4. The device of any one of claims 1-3, wherein the support wire is expandable in the axial direction.

5. The device of any one of claims 1-4, wherein the coiled structure 10 comprises different types of wire.

6. The device of any one of claims 1-5, wherein the support wire has a triangular or rectangular cross-section.

7. Method for supporting the wall of a lumen by inserting the device of claim 1 into the lumen, inducing the support wire to assume a predetermined shape, thereby expanding the device in the radial and/or axial direction.
